(19) United States
(12) Patent Application Publication
(10) Pub. No.: US 2010/0099944 A1
(43) Pub. Date: Apr. 22, 2010

(54) FECAL INCONTINENCE DEVICE, KIT AND METHOD

(76) Inventors: Tidhar Shalon, Tel-Aviv (IL); Guy Kotlizky, Kfar-Shemaryahu (IL)

Correspondence Address: MARTIN D. MOYNIHAN d/b/a PRTSI, INC., P.O. BOX 16446, ARLINGTON, VA, 22215, US

(21) Appl. No.: 12/527,867

(22) PCT Filed: Feb. 14, 2008
(86) PCT No.: PCT/IL08/00204

§ 371 Date: Aug. 20, 2009

Related U.S. Application Data
(60) Provisional application No. 60/902,090, filed on Feb. 20, 2007.

(51) Int. Cl. A61F 2/00

(52) U.S. Cl. 600/32

(57) ABSTRACT

A method for treating incontinence in a subject is provided. The method is effected by forming or positioning a plug within an incontinent passageway associated with the incontinence in the subject.

### FIELD AND BACKGROUND OF THE INVENTION

The present invention relates to devices and methods suitable for treating fecal incontinence.

Fecal incontinence (also referred to as anal or rectal incontinence) is a disorder afflicting both children and adults with devastating social and psychological affects. It is estimated that in the United States alone more than 5.5 million individuals suffer from fecal incontinence and that the incidence rate of this disorder is 1-5%. Fecal incontinence is a major factor limiting the rehabilitation of the elderly and disabled, preventing many of them from being cared for at home. Mild to moderate incontinence accounts for about 80% of the market (gas, liquid and soiling) while severe incontinence accounts for the remaining 20%.

Fecal continence results from a coordinated motor function of anal sphincters and pelvic floor muscles, the role of the rectum and sigmoid colon as a fecal reservoir with capacitance and compliance and as a propulsive force with intrinsic motor activity, the effects of stool consistency, volume and delivery rate, the anorectal angle, and anorectal sensation.

The most common causes of fecal incontinence are structural or functional deficiencies of the sphincter muscles. Such deficiencies can result from anatomic disruption of the sphincter mechanism which may be caused by obstetric injuries (perineal laceration and improperly performed median episiotomies), complications of fistula or fissure surgery (keyhole deformities), traumatic injuries (e.g., impalement injuries), or cancer or from deterioration of the sphincter muscles due to age, congenital disorders, systemic and metabolic diseases, acquired neurological defects, and diseases of the colon and rectum.

Anal sphincters are muscular structures that assist in controlling the flow of feces and release of flatus from the colon. The internal anal sphincter (IAS) and the external anal sphincter (EAS) encircle the anal canal and form a part of the anorectal ring (see FIG. 1). The IAS is a thickening of the gastrointestinal smooth muscle; it maintains continence at rest. The EAS is composed of striated, voluntary muscle. The EAS, the puborectalis, and the levator ani muscles work in concert to prevent leakage of flatus and feces when there is an increase in abdominal pressure or when the internal anal sphincter relaxes after rectal distention.

Resting pressure in the anal canal is typically 60 mm Hg (1.1 psi), increasing up to 100 mm HG (1.9 psi) in strain or forced exertion. In a normal individual (with the caveat that normal parameters vary widely), the myogenic activity of the involuntary internal sphincter contributes about 10% of the anal resting pressure, and 45% is due to the sympathetic innervation of the internal sphincter, for a total of 55%. The remainder of the resting tone is from the hemorrhoidal plexus (15%) and the external anal sphincter (30%). The external sphincter supplies 100% of the voluntary short-term squeeze pressure. Therefore, what is needed to improve continence is a technology that can influence the ability of the anus to seal better in the resting state while not interfering with the distention function of the sphincter during defecation.

Present treatment approaches for restoring fecal continence include non-surgical and surgical therapy. Non-surgical therapy for incontinence include biofeedback and perineal strengthening exercises beneficial in alleviating symptoms of seepage and occasional loss of control and electrical stimulation to improve contraction of the sphincter muscles.

Surgical therapy approaches include implantation of artificial valves (see, for example, U.S. Pat. Nos. 6,471,635, 6,749,556, and U.S. patent application Ser. Nos. 10/269,949 and 10/651,851), injection of bulking agents into the anal mucosa or the anal sphincters (see, for example, Vaizey and Kamm, British Journal of Surgery 2005; 92: 521-527), implanted electrodes for stimulating the pudendal or sacral nerves (see, for example, U.S. Pat. Nos. 6,907,293 and 7,054,689) or sphincteric muscles (see, for example, PCT publication No. WO06047833).

Although fecal incontinence can be partially controlled using absorbent pads, absorbent plugs or hard plugs/valves and the like, such approaches are not well tolerated by individuals due to efficacy, convenience and comfort limitations.

Thus, there remains a widely recognized need for a device and method for treating fecal incontinence which are devoid of the above limitations.

### SUMMARY OF THE INVENTION

According to one aspect of the present invention there is provided a method of treating incontinence in a subject comprising forming a plug within an incontinent passageway thereby treating incontinence in the subject.

According to further features in preferred embodiments of the invention described below, the incontinence is fecal incontinence and the passageway is an anal canal.

According to still further features in the described preferred embodiments the forming the plug is effected by administering a self-gelling or a self-solidifying liquid into the incontinent passageway.

According to still further features in the described preferred embodiments the forming the plug is effected by administering a liquid into the incontinent passageway and gelling or solidifying the liquid in the passageway.

According to still further features in the described preferred embodiments the plug is elastic.

According to still further features in the described preferred embodiments the plug is formed such that at least a portion of the plug is positioned below a pectinate line of the anal canal.

According to still further features in the described preferred embodiments the self-gelling liquid is an alginate preparation.

According to still further features in the described preferred embodiments the alginate is selected capable of forming an alginate foam.

According to still further features in the described preferred embodiments the self-solidifying liquid is a silicone preparation.

According to still further features in the described preferred embodiments the plug has a durometer hardness of 1 on the Shore 00 scale to 70 on the Shore A scale.

According to still further features in the described preferred embodiments the plug is a hydrophobic closed cell foam.

According to still further features in the described preferred embodiments the plug includes a gas permeable conduit.

According to still further features in the described preferred embodiments the plug is formed such that at least a portion of the plug is positioned above a pectinate line of the anal canal.

According to another aspect of the present invention there is provided a device for treating incontinence in a subject comprising an applicator head designed for administration of a preparation into an incontinent passageway, the preparation and the applicator head being configured such that the preparation gels or solidifies following administration into the incontinent passageway thereby forming a plug therein.

According to still further features in the described preferred embodiments the device further includes a mechanism for inducing gelling or solidification of the preparation.

According to still further features in the described preferred embodiments the mechanism is a mixing nozzle.

According to still further features in the described preferred embodiments the mechanism is a light source.

According to still further features in the described preferred embodiments the mechanism is a heat source.

According to still further features in the described preferred embodiments the device further comprises a reservoir for containing the preparation.

According to still further features in the described preferred embodiments the incontinence is fecal incontinence and the applicator head is designed for administration into an anal canal.

According to still further features in the described preferred embodiments the applicator head is designed such that at least a portion of the plug formed is positioned above a pectinate line.

According to still further features in the described preferred embodiments the applicator head is designed such that at least a portion of the plug formed is positioned below a pectinate line.

According to another aspect of the present invention there is provided a device for treating incontinence in a subject comprising an applicator head designed for administration of a fillable sac into an incontinent passageway, the sac and the applicator head being configured such that inflation of the sac following administration into the incontinent passageway plugs the passageway.

According to yet another aspect of the present invention there is provided a kit for treating incontinence comprising: (a) a preparation capable of gelling or solidifying to form an elastomeric body; and (b) a device comprising an applicator head designed for administration of the preparation into an incontinent passageway, the preparation and the applicator head being configured such that the preparation gels or solidifies following administration into the incontinent passageway thereby forming a plug therein.

According to still further features in the described preferred embodiments the preparation is an alginate, vinylpolysiloxane (VPS) or a silicone preparation.

According to still further features in the described preferred embodiments the device is configured such that the applicator head withdraws from the incontinent passageway during administration of the preparation.

According to still further features in the described preferred embodiments the applicator head is capable of conforming to a path of the incontinent passageway.

The present invention successfully addresses the shortcomings of the presently known configurations by providing devices and methods which are suitable for treating incontinent passageways.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates the anatomy of the anal canal and associated tissues.

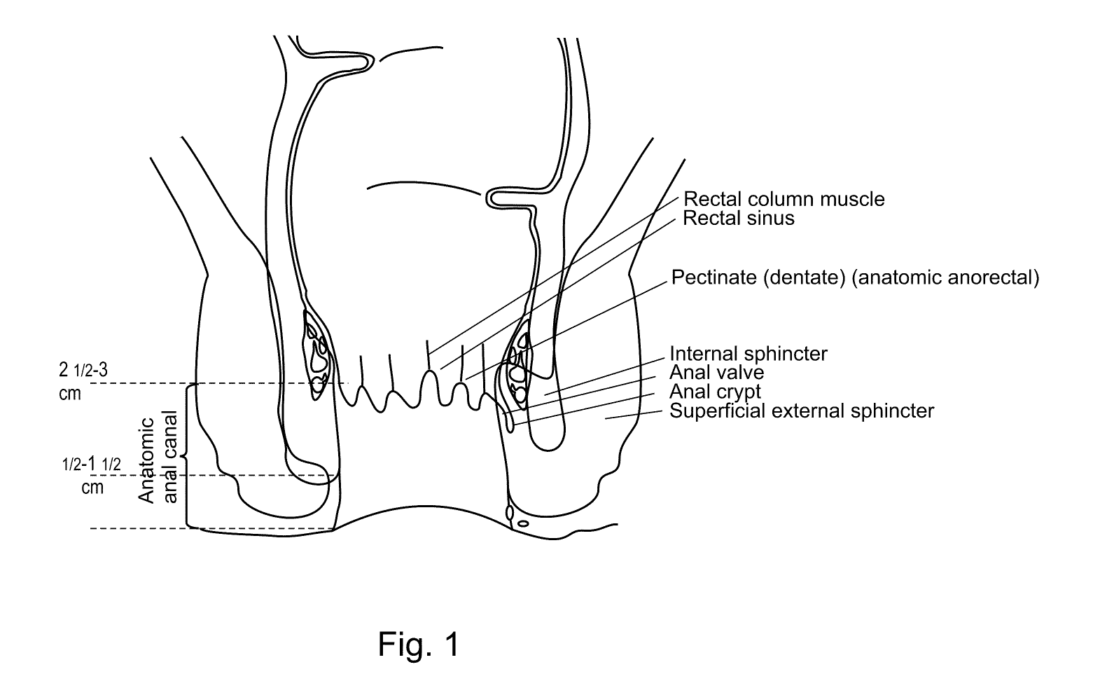

FIG. 2 illustrates an embodiment of a device for delivering a preparation into an incontinent passageway constructed in accordance with the teachings of the present invention.

FIG. 3 illustrates a silicone plug formed within an anal canal of a subject superimposed over an illustration of an anal canal.

FIG. 4a-c illustrate incontinence pads of a first incontinent subject prior to (FIG. 4a), during (FIG. 4b) and following (FIG. 4c) use of the present plug. One or more incontinence pads were collected for each day of the testing period.

FIG. 5a-c illustrate incontinence pads of a second incontinent subject prior to (FIG. 5a), during (FIG. 5b) and following (FIG. 5c) use of the present plug. One or more incontinence pads were collected for each day of the testing period.

### DESCRIPTION OF THE PREFERRED EMBODIMENTS

The present invention is of devices and methods which can be used to treat incontinent passageways. Specifically, the present invention provides a novel passageway plugging approach which can be used to treat fecal incontinence.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Fecal incontinence is a socially devastating disorder which affects at least 2.2 percent of community dwelling adults and 45 percent of nursing home residents. People who have fecal incontinence may feel ashamed, embarrassed, or humiliated; some don't want to leave the house out of fear they might have an accident in public.

Treatment of fecal incontinence depends on the cause and severity of the disorder.

Severe cases are typically treated by surgeries for repairing damaged sphincters, reinforcing anorectal structures, implanting artificial sphincters, and transferring muscle tissue. Mild to moderate cases of fecal incontinence are typically treated using special diets, medication, bowel training, or diapers. Although the latter approaches can reduce fecal discharge or help contain fecal discharge in some patients, they are either ineffective or are unacceptable for many patients.

Thus, despite the availability of pharmacological, behavioral and surgical treatments for fecal incontinence, many patients remain symptomatic.

To overcome the deficiencies of diapers and yet provide an easy non-surgical solution to this problem, use of anal plugs has been suggested. Such plugs are typically preformed from hard polymers or soft absorbent materials. The plug is introduced by the patient into the anal canal and much like a tampon is designed to contain or block any discharge.

Although such plugs have been proven somewhat effective in containing fecal discharges they are not well tolerated by patients (see, Deutekom and Dobben, “Plugs for containing faecal incontinence” 2007 The Cochrane Collaboration. Published by John Wiley & Sons, Ltd).

While reducing the present invention to practice, the present inventors have devised passageway plugging approaches which can be used to treat an incontinent passageway such as the anal canal while avoiding the above mentioned limitations of prior art anal plugs.

While experimenting with VPS plugs (see Examples 1 and 2), the present inventors have unexpectedly uncovered that plugs that form in the anal canal and thus form a negative impression of the tissue, strongly attach to the canal wall along the length of the formed plug due to the fact that the filling material flows into tissue folds and adheres to the tissue by virtue of a tight anatomical fit. This results in a plug that is anchored at more than one discrete location and is highly resistant to movement even in cases where the plug partially peels off the wall tissue due to movement of the rectal wall as the rectum fills with fecal matter. The present inventors have also uncovered that despite its strong anchoring, the plug is ejected effortlessly during voluntary defecation or can be removed manually thus allowing natural defecation when the need arises.

Thus, according to one aspect of the present invention there is provided a method of treating incontinence in a subject, such as a human.

According to one embodiment, the method of the present invention is effected by forming a plug against or within an incontinent passageway thereby treating incontinence in the subject.

As used herein, the term “passageway” refers to any biological conduit or duct through which a solid, liquid or gas is transported. Examples of passageways include the colorectal passageway, the urethral passageway, and parts of the alimentary passageway. The present invention is most suitable for use with the colorectal passageway and related incontinence problems. However, application thereof in treatment of, for example, urinary incontinence is also contemplated herein.

Incontinent passageways are passageways that are open beyond an optimal point and as such are characterized by reduced function. Passageway incontinence can be caused by disease, trauma to soft or hard tissues, anatomical deformities and the like. For example, in the colorectal passageway, damage to the pudendal nerve, the sphincteric or levator muscles can lead to incontinence of these passageways and to incontinence.

As used herein, the phrase “forming a plug” refers to in-situ forming of a plug-like structure within the incontinent passageway. Thus, the exact shape and form of the plug is formed within the passageway as opposed to the case where a preformed plug is pushed into the passageway.

Forming a plug within a passageway can be effected via any one of several approaches.

In a first approach a self-gelling/self-solidifying preparation is administered into the incontinent passageway as a liquid and allowed to gel or solidify.

In a second approach, a gellable or curable preparation is administered into the passageway and thereafter is induced to gel or solidify via application of energy in the form of light (e.g. U.V. light), pH, moisture, presence of ions, or heat.

In a third approach, a gellable or curable preparation is administered into the passageway and thereafter is induced to gel or solidify via administration of a gelling or curing agent.

Gelling or solidifying the preparation in the incontinent passageway provides several advantages.

First it ensures that the plug takes the form of the passageway in which it forms thus greatly reducing the chances of leakage or erosion of sensitive tissues.

Second, it enables control over the positioning of the formed plug. In the case of fecal incontinence, such positioning is critical for comfort since the anal canal (especially the pectinate line region) is sensitive to placement of foreign objects.

Preferably, the plug formed in the passageway is elastic such that it conforms to the shape of the passageway during movement thus being effective in sealing the passageway and comfortable in use over extended time periods (hours to days).

Typical hardness of such a formed plug can range between 1 on the durometer shore 00 scale to 70 on the durometer shore A scale, although higher or lower hardness can also be utilizes especially in cases where the plug is formed from more than one layers of material, or is formed over an underlying structure (further detailed hereinbelow).

The plug can also be formed such that it includes a conduit for gasses. In the case of fecal incontinence such a conduit can be used to release gasses while maintaining fluids and fecal matter trapped. Such a conduit can be formed by the preparation (e.g. a hydrophobic open-cell foam plug, or a hydrophilic open cell foam having pores selected of a size suitable for conducting gas and not liquids) once it is gelled or solidified, or can be generated in the formed plug via use of a dedicated applicator head (further described hereinbelow). However gas can also pass around a gas-impervious plug as is evident from the results presented in Example 2.

Several types of preparations which can be gelled or solidified in-situ can be utilized by the present invention to form a plug within an incontinent passageway.

The preparation can have a controllable adhesion to the tissue, as well as a controllable hydrophilicity, hydrophobicity, and hygroscopic properties. The preparation can be solid at room temperature when introduced into the rectum, liquefy at body temperature and then solidify again after a short time due to some polymerization reaction that was initiated by the heat, ph, pressure, moisture or other factor present in the body. The preparation can swell following insertion to improve the seal due to trapped gas in the injected material or to uptake of fluid from the surrounding tissues or lumen.

Specific examples of materials which can be used in the plug-forming preparation of the present invention are described below.

Alginates

Alginate is a linear polysaccharide, isolated, for example, from brown sea algae, which forms a stable hydrogel in the presence of divalent cations (e.g., Ba++, Ca++). Alginates occur naturally as copolymers of D-mannuronate (M) and L-gluronate (G) and have different monomer compositions when isolated from different natural sources. The block length of monomer units, overall composition and molecular weight of the alginate influence its properties. For example, calcium alginates rich in G are stiff materials, (see Sutherland, I W (1991): Alginates. In Biomaterials.: Novel materials from biological sources.). It is theorized that gel formation is due primarily to the G-block, and that the M-block is essentially non-selective. The calcium ions would thus be packed into the interstices between polygluronate chains associated pairwise and this structure is named the “egg-box” sequence. The ability to form a junction zone depends on the length of the G-blocks in different alginates. Advantages of alginates include their wide availability, low diffusional barrier for all nutrients and relative biocompatibility (Smidsrod et al., Trends in Biotech, 8:71-78, 1990).

Alginate can be mixed with additives such as, chitosan, diatomaceous earth, clay, silica and the like to provide further rigidity/elasticity; alginate can also be prepared as a foam (Alginate foam, Novamatrix). Such a foaming alginate preparation can have a pore size selected capable of absorbing liquids while serving as a conduit for gasses. Alternatively, the alginate can be introduced as two separate viscous liquids, one containing the alginate and the other containing the cross-linking agent which are mixed right before being introduced into the rectum, by a vortex mixing tip for example. Example alginate-based materials compatible with this invention are Earthium (f/x)tm two part life mold making materials (two part powder can be prepared as a stable two part solution and mixed right before application) or Artformtm mold making material by Lifecast (www.lifecast.net). Viscosity, rigidity and setting time can be fully controlled by the amount of water and ratio of the various constituents in these products.

A mixture of sodium and calcium alginates can be prepared that self-gel once mixed. See for example U.S. patent application 20060159823. In this embodiment, the two forms of alginates would be kept in separate compartments of an applicator and mixed, say with a vortex tip, while being introduced into the anal canal, thereby starting a polymerization process that forms a solid plug in situ within a minute or two.

Traditional sodium alginates can be gelled in situ using co-injection of the alginate and the polymerizing Ca++ ion (see for example Marler et. al., Plastic and Reconstructive Surgery, May 2000, 2049-2058).

Alginate preparations which can be used with the present invention are commercially available through FMC BioPolymers (USA) or Novamatrix (Norway).

Silicones

Silicones, or polysiloxanes, are inorganic-organic polymers with the chemical formula [R2SiO]n, where R=organic groups such as methyl, ethyl, and phenyl. These materials consist of an inorganic silicon-oxygen backbone ( . . . —Si—O—Si—O—Si—O— . . . ) with organic side groups attached to the silicon atoms, which are four-coordinate. In some cases organic side groups can be used to link two or more of these —Si—O— backbones together. By varying the —Si—O— chain lengths, side groups, and cross linking, silicones can be synthesized with a wide variety of properties and compositions

Silicone preparations which can be used with the present invention are commercially available from Zhermack SpA (Italy), examples include the Elite™ product line, in particular the light body, fast setting VPS which is preferred for its superior impression qualities.

Other materials which can be used with the preparation of the present invention include, but are not limited to, acrylate polymers (e.g. polyacrylamide), sodium hyaluronate; polyvinyl alcohol (PVA); hydroxypropylmethyl cellulose (HPMC) and its derivatives including hydroxyalkyl cellulose, sodium carboxymethyl cellulose, hydroxyethyl cellulose, hydroxypropyl cellulose, methylhydroxypropyl cellulose, methyl cellulose and ethylhydroxyethyl cellulose; thermoset elastomers, agar, polyurethane foam, and the like.

Denture materials can also be used by the present invention. Examples include, Super PoliGrip™ (active ingredients polyethylene oxide and sodium carboxymethyl cellulose), Polident™ which includes Polyethylene Oxide, Microcrystalline wax, Polybutene and Carboxymethyl-cellulose sodium and Effigrip™ which includes sodium carboxymethyl cellulose, gantrez and polyox. Other examples include the Dentsply™ denture relining material and the two component Luci-Sof™ Denture Liner System.

Additives that can be incorporated into the preparation include, but are not limited to, viscosity modifiers, gelation retarding agents, colorants, indicators, tackifiers, plasticizers, antioxidants, hydrocolloids (typically in the form of particles) and the like.

It will be appreciated that although the method of the present invention has been described in context with gellable/curable preparation, use of swellable desiccated hydrogels is also contemplated herein. Thus, the preparation can be a desiccated hydrogel that is inserted into the rectum as a rod of material like a suppository or as a solid wick, perhaps on a solid backbone and in an hour glass shape with the narrowest portion being in the hemorrhoid region, swells using the moisture in the rectum region and has low enough shear resistance that it molds itself into the space available in the rectum and anal canal during swelling. Examples of swellable hydrogels include desiccated polyacrylamide, desiccated alginate and the like. Alternatively, the material introduced can be a powder or a collection of particles. In the case of a cross-linkable material, for example alginate-based materials, the mixture can be introduced into the anal canal as a powder with the cross-linking agent present in the dry mixture, and the moisture from the tissue or lumen will eventually hydrate and gel the mixture. Alternatively, the powder can be dehydrated particles of material, either free to move around or captured in a matrix, that both soak up large quantities liquid, thus improving continence, and also swell to take up more room in the anal canal and rectum to provide physical blocking of the passage of liquid or solid fecal material. Therefore, the more liquid that is present, the more the particles gel or swell to accommodate the natural and dynamic shape of the anal anatomy.

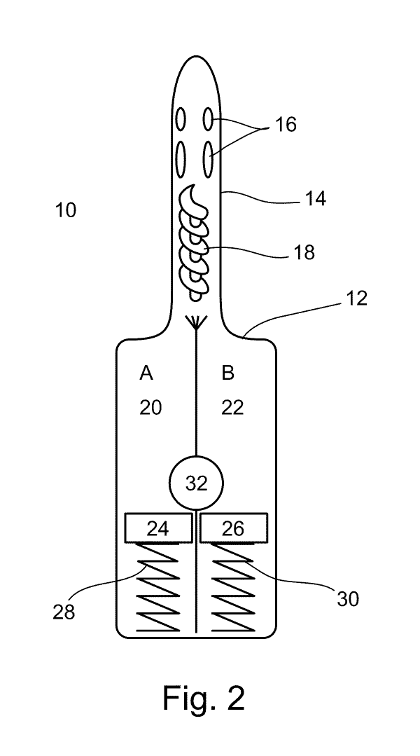

The plug forming preparation of the present invention can be delivered using any device suitable for intra-passageway delivery, including, a syringe, a delivery catheter and the like. FIG. 2 illustrates an embodiment of a dedicated delivery device which is referred to herein as device 10. Device 10 is designed for forming a plug within an anal canal, over a region spanning the anal canal to a point below the pectinate line or extending throughout the anal canal and into the colon above the hemorrhoids.

As is shown in FIG. 2, device 10 includes an applicator head 12 which can include a delivery tube 14 having a vortex mixer 18 (for example, the Devcon Permatex mix nozzle, Devcon USA) and holes 16 for forward, sideways or rearward delivery of a two-part gellable/curable preparation A and B in separate compartments 20 and 22 pushed through delivery tube 14 by pistons 24 and 26 which are in turn push by springs 28 and 30 which is released by push button trigger 32. Such a delivery approach can also be used with alginates, wherein a sodium alginate solution can be kept in compartment 20 and a calcium ion containing solution in compartment 22.

Gas formed in the body can be released between the formed plug and the anal canal wall (as is evident from the results presented in Example 2), or else through a dedicated gas passageway formed in the device itself Such a passageway can contain a deodorant or absorbent such as activated charcoal to minimize the odor associated with the gas.

A dedicated gas passageway can be a gas tube (not shown) which is positioned within tube 14 and is delivered along with the preparation into the anal canal. Such a gas tube can be about 2 mm in diameter; it can be designed for delivery with the preparation (via for example, an ejector mechanism which is actuated with the release of the preparation). Once the preparation gels or solidifies, the gas tube forms a backbone or spine of the plug and allows for gas venting. A string 18 can be attached to the second tube to allow for removal (like a tampon) of device 10 if need be before it will be naturally ejected while defecating.

Alternatively, the delivered preparation can be composed of a material that gels/cures to form an elastomeric and hydrophobic open cell foam that lets gas pass through while blocking liquid or solid materials, an example of such a foam is polyurethane.

An alternative configuration of applicator head 12 can include the use of a nozzle embedded with a dissolvable cross linker (e.g. calcium chloride) and a reservoir filled with the polymerizable material. Pushing the polymerizable material through the nozzle initiates the cross linking while the preparation is delivered into the anal canal.

Yet another alternative approach can include the use of a cross linking agent embedded in a thin water-soluble membrane (e.g. a material similar to Listerine Cool Mint™ Oral Care Strips) with the material to be cross linked in a powdered or non-aqueous solvent (such as DMSO). Upon insertion into the rectum, the moisture in the tissue or lumen will dissolve the outer membrane and release the cross linking agent to diffuse into the material to be cross linked. Another method of releasing the cross linker can involve the breakdown of a heat-sensitive barrier membrane that is intact at room temperature but degrades at body temperature.

Pre-delivery gelling/curing can be effected via a mixing chamber which includes the components of the preparation separated until mixing. This can be achieved via a membrane/partition which can be ruptured broken via shaking or mechanical agitation.

Alternatively, such a mechanism can be a heating chamber for melting/dissolving the preparation (e.g. agar) and cooling it down to a temperature of delivery (e.g. 41° C. for agar). The sol-gel transition temperature of a preparation can be controlled (e.g. lowered) by use of additives. For example, the sol-gel transition temperature of agar can be lowered to 36° C. using 0.3-0.4% sodium lauryl sulfate (SLS).

As is mentioned hereinabove, accurate placement of the formed plug is critical for both comfort and function.

The formed plug should reside in the anal canal, either below the pectinate line which includes the sensors activating bowl movement or above it. The formed plug should remain in tight contact with the walls of the anal canal such that it hardly perceivable by the user. The formed plug does not need to be very hard, as it only needs to prevent seepage of solids, liquid and gases. Yet at the same time, the plug should be solid enough not to break apart or dislodged with the passage of gas or normal motion of the anal canal. Therefore a minimum consistency would have about 1 million centi-Poise viscosity—roughly equal to Crisco™ shortening or soft peanut butter and a maximum hardness would be equivalent to silicone with a hardness of 70 on the Shore A scale.

Device 10 further includes a mechanism for controlling deposition of the preparation within the anal canal such that accurate placement of the formed plug is achieved. The material or materials in device 10 can be pressurized with a spring loaded piston or gas pressure, or driven via a motor so that the user would not need to apply force to introduce the materials into the rectum, but rather only need to release a trigger or open a valve. Device 10 can be designed so that the force applied by the user to activate the trigger or valve is greater than the force required to insert the tip of device 10 into the anal canal (albeit in the same direction), thereby assuring a properly seated device before the material is released into the rectum.

Device 10 can further include a simple stop on delivery tube 14 for controlling depth of penetration of delivery tube 14.

Preferably, device 10 is designed with a manually-operated or automatic mechanism (not shown) to coordinate the injection of preparation A and B during the withdrawal of delivery tube 14 from the anal canal, thereby making room for the preparation and ensuring uniform delivery of the preparation in the anal canal. Such a function of device 10 can be effected using one of several approaches. Deposition of the preparation and withdrawal of delivery tube 14 can be coordinated such that the preparation is not deposited outside the anus, alternatively, a dab of material or a withdrawal element can be left behind outside the anus to facilitate removal of the formed plug if need be.

Device 10 is used as follows, delivery tube 14 is pushed 2-5 cm into the anal canal through the rectum, the tip of delivery tube 14 can be self lubricating to enable penetration or it can be lubricated prior to use. Once positioned, the material contained in a reservoir of device 10 is automatically mixed (if need be), delivered and allowed to gel/cure. Device 10 can include a timer and alarm for indicating time of removal of device 10 from the anal canal. Once the tip of delivery tube 14 is inserted into the anal canal, administration of the preparation can be rapid and complete enough such that by the time the device is pulled out of the anal canal, no material is being pushed out of the tip. This ensures that the skin outside the anal canal remains material-free. Alternatively, device 10 can include a scraper for removing excess plug material from around the rectum.

The tip of delivery tube 14 can have a cone shaped soft detachable plug (e.g. fabricated from a polymer) that allows for painless insertion; the tip can detach from the applicator when the preparation is pushed out of delivery tube 14 and adhere to the plug formed and thus excreted together with the plug. Such a “nose cone” can have a string attached to it running down the length of the applicator, either inside or outside it. The string would run through the resulting plug and emerge outside the anal canal and allow a user to pull the plug out before normal bowel evacuation if desired. The tip of delivery tube 14 can be rounded with petal-shaped tips (like a tampon inserter or can of whipped cream) to allow for easy insertion and flow of the preparation through the petals. Delivery tube 14 can be arched backwards to more readily match the curved geometry (angulation) of the anal canal (as viewed in a sagittal section) and therefore follow the path of the canal and ease insertion. Alternatively, it can be fabricated from an elastic/plastic material and assume the curved shape upon insertion. In any case, the length of delivery tube 14 ranges from 7 to 12 cm.

Device 10 can form a part of a kit which also includes the preparation to be administered. The kit can further include instructions for use in self applying the preparation to an anal canal.

Device 10 can be disposable and preloaded with a standard volumes of the preparation, or else the volume can be set by the user. Device 10 can be reusable and contain a pressurized multi-dose amount of the preparation and have a disposable tip or shield only. The preparation would be released in a metered dose format once per use.

Device 10 can also be a foil or polymer pack which includes a nozzle and compartments for keeping the two components separate and for allowing such components to mix upon delivery. Mixing can occur in a tortuous nozzle fabricated by heat sealing two layers of film in a labyrinth pattern. The two components can be squeezed out of the foil pack using a roller, and the pressure generated by such squeezing can also stiffen the mixing portion of the foil pack sufficiently to enable it to be inserted into the anal canal. Further pressure applied by the roller would then puncture the tip of the nozzle and release the material into the anal canal. Rolling up the foil packaging would withdraw the mixing nozzle from the anal canal while at the same time squeezing the preparation out of the nozzle into the anal canal.

According to another aspect of the present invention, device 10 can introduce particles, say 0.5-3 mm. diameter silicone balls, into the anal canal and rectum that would stick together based on their surface chemistry sufficiently to form a form fitting anal plug.

According to another aspect of the present invention, device 10 can include a thin inflatable sac that can be inflated with gas, liquid or a gel/solid to assume the shape of the inner volume of the lumen of the anal canal. Example materials for such a sac are latex, silicone, polyurethane, and other polymers with a thickness in the range of 15-100 microns. Example filling materials are air, saline, Aloe Vera gel, glycerin, wax, oil, silicones, foams, pastes sticky microparticles (Floam™) or any of the substances described herein.

The advantage of this embodiment of device 10 is that the sac can shield the mucosa of the anal canal from filling material which could irritate mucosal surfaces as well as contain filling material which cannot be maintained in the anal canal otherwise. Such a sac could be introduced using a rigid hollow insertion tube. The filling material would inflate and fill the sac through the insertion tube and through a one-way valve or septum and then the sac can be detached from the filling reservoir. Filling is preferably effected following insertion of the empty sac into the anal canal, however, prefilled sacs that are inserted in a deformed/folded state are also envisaged.

The shape of a filled sac could resemble an upside-down cone. A removal string can be attached to the cone tip to facilitate removal of the device. The outer surface of such a sac would be pliable and smooth enough to conform to the inner folds of the mucosal surfaces of the rectum.

The sac can alternatively include pores that allow the filling material to exude out of the sac during filling thereby enabling the formation of a thin layer of material on the surface of the sac. Such a configuration would be advantageous in cases where the filling material is a viscous flowable material or gel such as Aloe Vera.

The administered substance or an outer surface of device 10 can elute an active compound, such as glycerin laxatives, an anti-hemorrhoid agent such as PreparationH™, or colitis or fistula treatment agents.

The methodology and devices described herein can be used in a diagnostic procedure for preparing a cast of the inner volume of the anal passageway to help diagnose the causes and recommend treatment of fecal incontinence, much like a dentist performs a dental impression to plan an orthodontic procedure.

Thus, the present invention provides devices and methods for treating incontinent passageways and in particular mild to severe cases of fecal incontinence. The devices and methods of the present invention provide the following advantages:

(i) the plug conforms to tissue thereby resulting in a tight seal and reduced discomfort when in use;

(ii) in-situ gelling/curing/swelling/sealing ensures a personalized fit;

(iii) elasticity and perfect anatomical fit of the plug, as well as conformity to surface morphology reduces migration and discomfort, while maintaining seal and yet allowing flatulence to escape around or through the formed plug; and

(iv) easy and complete removal with defecation.

It will be appreciated that materials utilized by the present invention to form plugs within the anal canal can also be used in a similar manner to plug other body passageways. For example, an external plug formed over the urethral opening of the urethra can be used to provide continence, especially to stress urinary incontinence patients. Such a formed plug can take the shape of a flat disc which can be formed by simply dabbing a small amount of the material (e.g. VPS) over the opening of the urethra. Since material such as VPS can strongly adhere to skin due to its ability to form within creases and cracks in the skin surface, an external plug formed from, for example, VPS would not fall off accidentally or and yet can still be removed easily via peeling.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following example, which together with the above descriptions, illustrate the invention in a non limiting fashion.

### Example 1

### VPS plug

VPS is a two part material that hardens over a short period of time, creating an elastic product. This substance is FDA and CE approved for dental use, and is used routinely on a global basis in dentistry to make dental impressions. The substance does not stick to the tissue, and therefore is removable therefrom. The components of VPS are approved for use in humans and no complications or risks are involved in medical use of VPS.

Two milliliters of vinylpolysiloxane (Zhermak Elite HD) were self administered into the anal canal of a continent male subject using a mixing nozzle (Zhermak) which mixes the preparation as it is pushed out of the nozzle. The mixing nozzle was covered by a standard hollow enema plastic tip to protect the anal canal from the sharp end of the mixing nozzle. The applicator was withdrawn from the anal canal during injection through the mixing nozzle and the injected material cured within 4 minutes. The subject carried the plug for 24 hours.

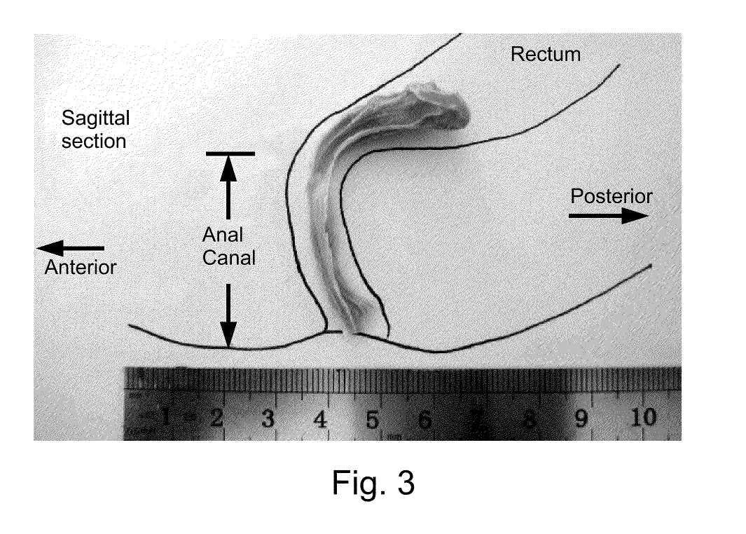

Following the 24 hour period, the formed plug was ejected with defecation and collected. FIG. 3 illustrates the formed plug superimposed over an illustration of anal canal anatomy. As is shown by this Figure, the plug formed along the length of the anal canal from the rectum to a region 2-3 cm above the hemorrhoids.

The subject reported that the formed plug did not induce any perceived discomfort and remained in position throughout the experiment. The subject also reported that flatulence escaped around the plug without inducing any resistance to gas release or perceivable dislocation of the plug or leakage.

### Example 2

### Study Testing Efficacy of a VPS Plug

Using the methods of Example 1, VPS was injected into the anal canal of two women patients—the first a Wexner 20 patient and the second a Wexner 17 patient in order to assess the functionality of an in-situ forming anatomical anal plug. The higher the Wexner score, the less continent the patients are. In this case, both patients were severely incontinent as 20 is the highest possible Wexner score. Both women were evaluated for a week prior to use of the plug, over two weeks with daily injections of a plug and for a week following plug use. One or more incontinence pads were collected for each day of the study.

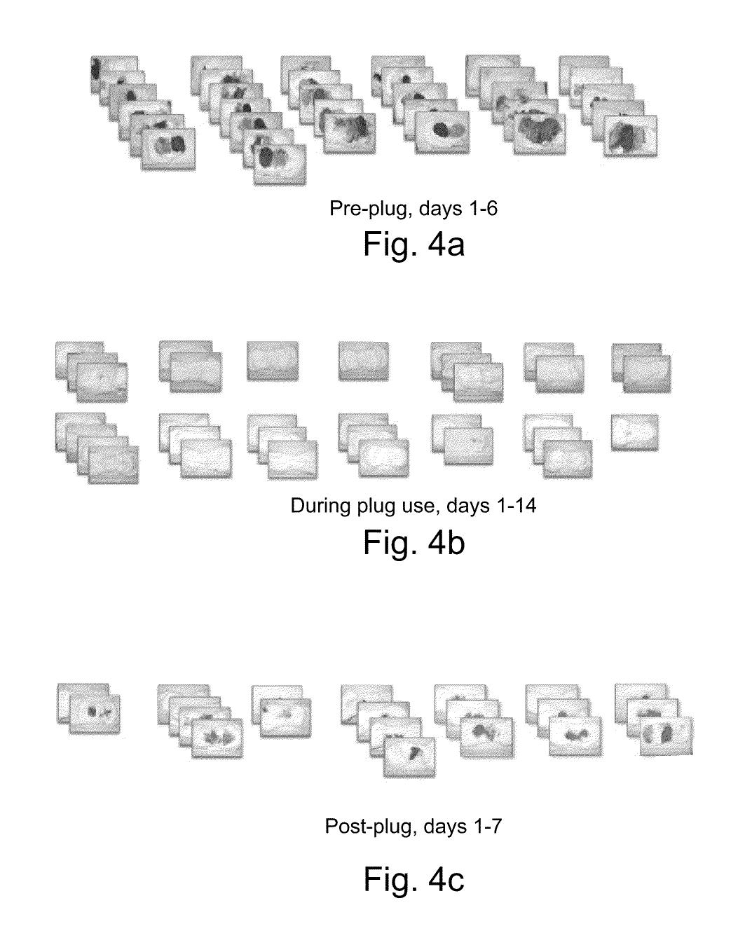

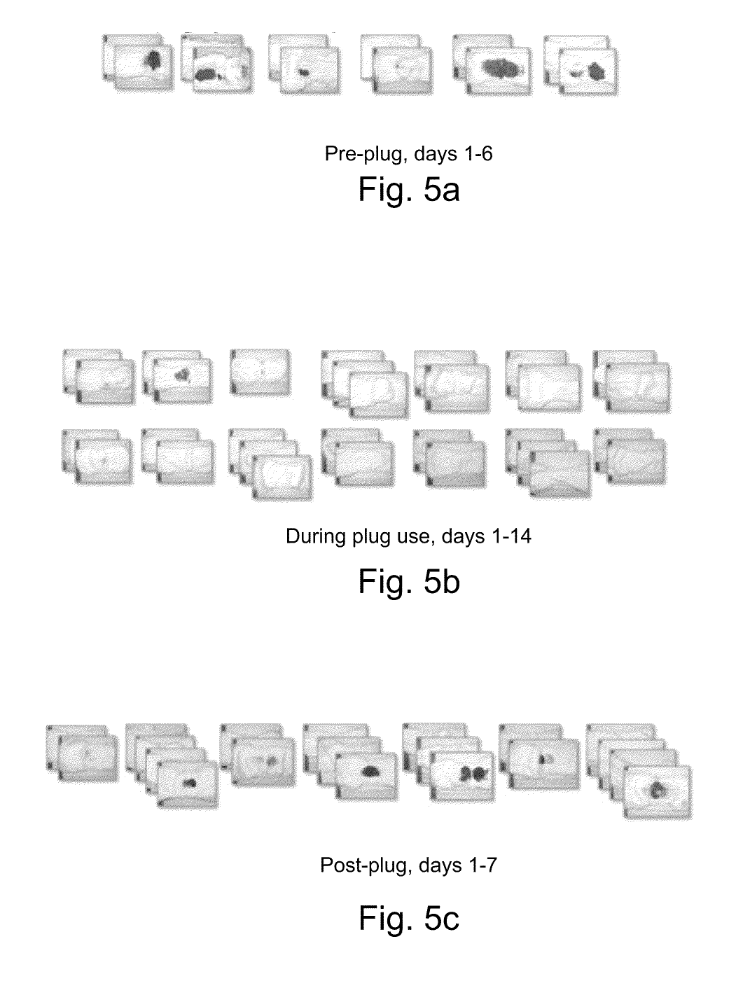

Prior to plug injection, both patients had no practical control over their defecation (FIGS. 4a and 5a). During the two weeks of plug use, VPS was injected once a day, between 06:00 and 08:00 every morning, following natural defecation. The patients had no problem maintaining the plug in position during this 24 hour period, and to naturally eject the plug as an integral part of normal voluntary defecation.

During the 14 day plug injection period, uncontrolled defecation events had practically disappeared in both patients (FIGS. 4b and 5b). It should be noted that the pads were replaced a few times a day as part of the experiment and not because of soiling. When the plug was in place, both patients reported full control over flatulence without uncontrolled solid and/or liquid defecation.

No adverse events were recorded in both patients. Additionally, although both patients slightly sensed the presence of the plug during the first two days of the study, no plug sensation was reported for the remaining days of the study. Both patients reported their strong desire to continue using the plug and indicated that it dramatically improved their quality of life.

Following the 14 days of plug use, both patients sopped getting injected VPS plugs and returned to using pads only. As is shown in FIGS. 4c and 5c, both patients exhibited uncontrolled soiling.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-14. (canceled)

15. A method of treating incontinence in a subject comprising administering a liquid or gel capable of gelling or solidifying in an incontinent passageway and forming a plug therein thereby treating incontinence in the subject.

16. The method of claim 15, wherein the incontinence is fecal incontinence and said passageway is an anal canal.

17. The method of claim 15, wherein said liquid or gel self-gels or self-solidifies within said incontinent passageway following said administration.

18. The method of claim 15, wherein said plug formed in said incontinent passageway is elastic.

19. The method of claim 17, wherein said liquid or gel is a polymer preparation.

20. The method of claim 17, wherein said liquid or gel is a polysiloxane preparation.

21. The method of claim 15, wherein said administering is effected by positioning a fillable sac within said incontinent passageway and filling said sac with said liquid or gel prior to or following positioning.

22. A kit for treating incontinence comprising:
(a) a preparation capable of gelling or solidifying; and
(b) a device comprising an applicator head designed for administration of said preparation into an incontinent passageway, said preparation and said applicator head being configured such that said preparation gels or solidifies following administration into said incontinent passageway thereby forming a plug therein.

23. The kit of claim 22, wherein said preparation is a polymerizable preparation.

24. The kit of claim 22, wherein said device further includes a mechanism for inducing gelling or solidification of said preparation.

25. The kit of claim 24, wherein said mechanism is a mixing nozzle.

26. The kit of claim 22, further comprising a sac attached to said applicator head, said sac being positionable within said incontinent passageway for containing said preparation administered from said device.

27. The kit of claim 22, wherein said sac is filled with said preparation prior to being positioned within said incontinent passageway.

28. The kit of claim 22, wherein the incontinence is fecal incontinence and said applicator head is designed for administration into an anal canal.

29. The kit of claim 22, wherein said device is configured such that said applicator head withdraws from said incontinent passageway during said administration of said preparation.

30. A device for treating incontinence comprising a sac sized and configured for positioning within an incontinent passageway, said sac being filled with a liquid or gel.

31. The device of claim 30, wherein said liquid or gel is capable of gelling or solidifying.

32. The device of claim 30, wherein the incontinence is fecal incontinence and said passageway is an anal canal.

33. The device of claim 31, wherein the incontinence is fecal incontinence and said passageway is an anal canal.
